(19) United States
(12) Patent Application Publication
(10) Pub. No.: US 2006/0004096 A1
(43) Pub. Date: Jan. 5, 2006

(54) Method of Treating Endothelial Dysfunction, Oxidative Stress and Related Diseases

(76) Inventor: Joseph Larner, Glen Allen, VA (US)

Correspondence Address: WILLIAMS MULLEN;ENTERPRISE CENTER, 401 E MARKET ST, SUITE 101, CHARLOTTESVILLE, VA, 22902-5264, US

(21) Appl. No.: 10/908,800

(22) Filed: May 26, 2005

Related U.S. Application Data
(60) Provisional application No. 60/575,740, filed on May 28, 2004.

(51) Int. Cl. A61K 31/225

(52) U.S. Cl. 514/548

(57) ABSTRACT

A composition, either as a nutritional supplement or pharmaceutical, for the treatment of oxidative stress, endothelial dysfunction and related disease states which comprises administration of D-chiroinositol (DCI) congeners, acting as an antioxidant or glucose uptake promoter and metabolic normalizer, is disclosed. A composition of treating oxidative stress comprising administration of DCI is also disclosed. The administration of DCI derivatives comprises administering to the whole animal a dose in an amount sufficient to normalize blood glucose and triglycerides and to ameliorate endothelial dysfunction. The administration can be an oral, injectable, intranasal, or patch dosage forms. DCI is found in the food chain and has been shown to be very safe in large doses and, therefore, the amounts sufficient to achieve the desired therapeutic antioxidant effect will be low relative to the amounts reaching toxic levels. Therefore, DCI can be administered orally as a prophylactic nutritional supplement.

Oxidative stress is a cellular metabolic disorder that contributes to the pathophysiology of a large number of disease states. The connection between oxidative stress and these disease states and particularly insulin resistance has been the subject of much inquiry in recent times.

Insulin resistance is the inability of insulin to promote glucose uptake in muscle and fat. It can be due to inherited or acquired causes or a combination of the two. There are some clear mutations and abnormalities, including defects in insulin receptors, glucose transporters, or some of the signaling proteins. On the other hand acquired factors also contribute to the onset of insulin resistance including inactivity and overeating and aging. The use of some medications such as steroids, sometimes beta-blockers, and diuretics can also increase insulin resistance. Hyperglycemia by itself is toxic to insulin action; this is a concept known as glucotoxicity. And the elevation of free fatty acids, also known as lipotoxicity, now is known to also decrease insulin action in muscle and fat. A combination of all these abnormalities is responsible for the development of insulin resistance.

Syndrome X is a term that is used to describe a group of disease states that is related to insulin resistance. Physiological conditions known in the art to be associated with insulin resistance include diabetes, arthrosclerosis, hypertension, AIDS, cancer, sepsis, aging, lupus, obesity, hyperinsulinemia, hypertriglyceridaemia, dyslipidemia, hypercholesterolemia, polycystic ovary syndrome (PCOS), hyperandrogenism, anovulation and endothelial dysfunction. Endothelial dysfunction exacerbates insulin resistance and beta cell dysfunction, so much so that people with diabetes with no history of cardiovascular disease (CVD) are at as much of a risk of suffering from CVD as people who have had one CVD episode.

The clinical sequelae of endothelial dysfunction is typically hypoxia, ischemia and reperfusion leading to oxidative stress and, ultimately, to CVD. The risk factors that have been linked to CVD (i.e., high cholesterol, hypertension, smoking, heart failure, and particularly diabetes) are now known to cause endothelial oxidative stress. The resultant endothelial damage leads to superoxide production and oxygen free radicals, and this then leads to endothelial dysfunction.

The mitochondria are the major sites for the production of oxygen free radicals and other reactive oxygen species (ROS). The mitochondrion is the primary site of oxidative metabolism where ATP production is linked to the coupling of electron and proton transfer across the mitochondrial membrane. Inside the mitochondria a pool of electron carrier molecules (e.g., NAD, FAD) facilitate the movement of electrons for the production of ATP. Outside the mitochondria an excess of protons help stabilize the membrane potential. Insulin resistance related oxidative stress disrupts the coupling between electron and proton transfer, resulting in a reduction in the electron carrier pool. The resultant higher electron potential within the mitochondrion unleashes the electrons onto indigenous oxygen generating superoxide anions.

Oxidative stress usually develops when the pro-oxidant action of an inducer exceeds the anti-oxidant capacity of the cell defense system, altering its homeostatic capacity. High levels of glucose and fatty acids act as pro-oxidants and although the exact mechanism is not known, it is believed that they increase the mitochondrial electron potential that leads to unusually high superoxide production. Studies have implicated iron as the catalyst for the increased electron output in the hyperglycemic condition.

A blood vessel with a normal endothelium (i.e., good vascular tone) retards platelet leukocyte adhesion, inhibits smooth muscle cell migration and serves as a barrier to low-density lipoprotein (LDL). Oxidative metabolism within the normal vessel walls exists as a balance between superoxide anion and NO production. But when that endothelium is injured by the very risk factors causing oxidative stress, superoxide production is increased causing breakdown of NO, which stimulates integrin expression promoting platelet and leukocyte adhesion to the endothelium, leading to an inflammatory response. The superoxide anion also inactivates NO, causes cellular proliferation, alters kinase activity and facilitates lipid peroxidation. Smooth muscle cells begin to migrate, which remodels the vessel and increases lipid deposition. All of these deleterious events lead to a developing atherosclerotic plaque and vasoconstriction.

There are methods to treat diabetic manifested endothelial dysfunction and the drugs typically used are directed to reduce the damaging effects of hyperglycemia (metformin, TZD's and sulfonylureas), high cholesterol (statins), hypertension (ACE inhibitors and Ca2+ channel blockers), hyperlipidemia (Gemfibrozil), fibrinolysis (aspirin) and thrombosis. It should be noted that the TZD's and metformin are the only class of drug on the market that is directed toward increasing targeted cells sensitivity to insulin's action.

Clearly oxidative stress is important in the pathology of insulin resistance where the mechanism of its deleterious effects resides mostly in the mitochondria of cells associated with the metabolic pathways of glucose and fatty acids. Since ATP production is so closely linked to proper oxidative metabolism one would expect that oxidative stress could impact the function of virtually every organ in the body. Thus, it should not be surprising to learn that oxidative stress is also associated with the pathology of other metabolic disorders.

Iron's ability to facilitate the progression of oxidative stress has lead many researchers to discover the key role that this important mineral has on the etiology of oxidative stress in some diseases. Intravenous administration of iron, typically as iron dextran for the treatment of anemia, has been linked to increased oxidative stress. Co-administration of an antioxidant as adjunct therapy to IV iron for treatment for anemia, kidney disease and related disease states has been proposed.

Hepatic fibrinogenesis which is the final common pathway for a variety of chronic liver diseases has been linked to iron overload and high levels of dietary fat. These dietary abnormalities are integral to the effects that oxidative stress has on the progression of hepatic fibrinogenesis.

Iron accumulates in the brain as we age. Although the link between oxidative stress and Alzheimer's disease has not been clearly established, significant mitochondrial disorganization and decreased mitochondrial DNA have been reported in patients with Alzheimer's disease. Thus oxidative stress and iron overload are implicated in the progression of Alzheimer's disease.

Parkinson's disease is caused by the degeneration of the substania nigra in the brain, where large stores of iron are found. The most prescribed treatment for Parkinson's disease is levodopa, which is actually a prodrug that is metabolized to dopamine in the brain. It has been shown that levodopa generates reactive oxygen species through oxidative metabolism of the conversion of levodopa to dopamine. It is reasonable to conclude, therefore, that iron linked oxidative stress, is a contributing factor to the pathogenesis of Parkinson's disease.

In some cases iron has not been found to contribute to the progression of oxidative stress related diseases. Perhaps other cofactors are involved but nevertheless oxidative stress contributes to the progression of many other disease states. For example, a marker for oxidative stress related modification of the protein structure of collagen has been shown to increase with advanced lumbar disc degeneration. Also plasma membrane integrity loss is integral to necrosis (but not apoptosis). Oxidative stress induces necrotic cell death in the gut and is linked to the pathogenesis of gut derived disease states such as Crohn's disease.

There is an association between erectile dysfunction (ED) and cardiovascular disease. The connection is in the cavernosal endothelial cells, which have been shown to contribute to pro-erectile nitric oxide production, an important factor in normal erectile function. It has been suggested that abnormalities in this function may be an early clinical manifestation of cardiovascular disease and that both ED and CVD are linked to oxidative stress.

Further examination of the medical literature reveals that oxidative stress is involved in the pathogenesis or progression of fibriotoxicity, chronic pancreatitis, glaucoma, muscular dystrophy, fibriomyalgia, rheumatoid arthritis, amyloid lateral sclerosis, wound healing, spermatozoa damage, high blood pressure, cancer, cataracts, asthma, nonischemic cardiopathy, exercise intolerance in patients with heart failure and arterial fibrillation. There are probably many other diseases that can be added to this list.

There have been studies to suggest that vitamin E, vitamin C, Coenzyme Q, α-lipoic acid and other known antioxidant nutritional supplements are all effective preventive treatments. For example, Vitamins E and D, selenium, phytoestrogens/isoflavaones and lycopene may play a role in the chemoprevention of prostrate cancer. In addition, peripheral arterial disease responds to glutathione and N-acetylcysteine decreases cytokines and the gene expression of other nuclear activation factors that are responsible for tissue damage from sepsis conditions. Vitamin E may be the most used anti-oxidant nutritional supplement and its capacity to destroy free radicals has been demonstrated both in vitro and in vivo.

Vitamin E has been suggested to be important in the amelioration of many diseases of the circulatory system. Cardiologists have recommended the use of vitamin E as a prophylactic treatment for heart attacks, angina, atherosclerosis, rheumatic fever, acute and chronic rheumatic heart disease, congenital heart diseases, intermittent claudication, varicose veins, thrombophlebitis, and high blood pressure. The list of purported benefits of vitamin E is long and includes an oxygen-sparing effect on heart muscle, helping to gradually break down and prevent blood clots in the circulatory system, encouraging collateral circulation in the smaller blood vessels of the body, promoting healing with the formation of much less scar tissue, helping to strengthen and regulate the heartbeat, helping to protect the immune system by strengthening the cell membranes against oxidation, aiding in the formation of muscles and other tissues, and shielding other nutrients such as Vitamin A and C.

Myoinositol hexaphosphate, which is also known as phytic acid, is sold commercially as a naturally occurring antioxidant. Studies have shown that the antioxidant mechanism of phytic acid may be its ability to inhibit the generation of ROS from hydrogen peroxide by chelating transition metal ions. In fact, high concentrations of phytic acid chelate intestinal iron thereby preventing it from participating in the generation of ROS in that milieu. Molecular modeling studies have shown that phytic acid occupies all six coordination sites in the ferric phytate complex. This is important because even chelated iron is catalytically active if there is an available coordination site. Furthermore, this complex shifts the redox potential of iron such that ferrous iron formation is prevented and cannot participate in the Fenton reaction, which is responsible for hydroxyl radical production.

Myoinositol has been shown to decrease the peroxidation effect of hydrogen peroxide in human erythrocytes and human cataractous lenses. The antioxidant property of inositol was attributed to its quenching of ROS and its conjugation with arachidonic acid and glucose. The antiglycating properties of inositol have important implications in the oxidative stress associated pathogenesis of insulin resistance as described above.

Some of the antioxidant nutritional supplements can act as pro-oxidants in certain circumstances. Vitamin A, vitamin E, quercetin, and N-acetylcysteine all have shown to cause oxidative DNA damage. Phytic acid, on the other hand, does not cause oxidative DNA damage even at high concentrations. Thus DCI and its congeners are also useful as an antioxidant that does not have pro-oxidant potential.

Whereas the nutritional and health benefits of Vitamin E, phytic acid and other vitamins and minerals have been demonstrated, their administration is not often used as a first line of therapy for serious disease states related to oxidative stress. Indeed when insulin resistance is considered, the nutritional supplements currently on the market do not seem to direct their action to that particular metabolic disorder. Thus there exists a need for a nutritional supplement, and therapeutic agent directed to the disorders that are associated with insulin resistance and the concomitant oxidative stress. In addition, as in the cases of Vitamin E and phytic acid, the administration of such a nutritional supplement or therapeutic agent can be important in the treatment of oxidative stress in general and all of the disease states associated with oxidative stress.

The mechanism of insulin signaling has been studied by Larner, et. al., and other research groups and it was through this research that a unique class of inositol glycans was identified. These inositol glycans, which were isolated from membrane phospholipids or proteinated inositol glycan species activate key enzymes involved in the biochemical signaling cascade associated with insulin's metabolic function. Thus they mimic some of the cellular metabolic actions of insulin to dispose of glucose. One specific member of this class of inositol glycans, which contains D-chiroinositol (DCI) and galactosamine, activates key intracellular rate limiting enzymes of oxidative and non-oxidative glucose metabolism. In vivo this DCI containing glycan rapidly lowers elevated blood glucose to normal, with no hypoglycemia, when administered intravenously to diabetic rats.

The amount of DCI in diabetic human urine and muscle, diabetic monkey urine, genetic G/K type 2 diabetic rat urine, muscle, liver and kidney was lower than that found in normal individuals. A related inositol, myoinositol was increased relative to normal individuals in these studies. The decrease in urine DCI in humans and monkeys was inversely correlated to the degree of insulin resistance in the respective subjects. The DCI research taken together implicates DCI as an important factor in the etiology of insulin resistance.

The use of DCI and its derivative for the treatment of a variety of metabolic disorders, more specifically those that are related to insulin resistance have been the subject matter of patents and publications. The treatment of insulin resistance in humans by the administration of DCI was the subject matter of U.S. Pat. No. 5,124,360. Since DCI is found in the food chain it has a safety profile that warrants its use as a nutritional supplement. A method of treating a cluster of diseases with elevated blood sugar by the administration of a dietary supplement of DCI was the subject of U.S. Pat. No. 5,428,066. There have been several publications reporting that DCI administered to STZ type 2 diabetic rats, monkeys and humans demonstrated effective decrease of hyperglycemia, triglyceridemia and enhanced glucose disposal. DCI was effective in human PCOS in restoring ovulation, glucose tolerance and metabolic balance. More recent results showed that chronic administration of DCI (20 mg/kg 2× daily for 4 weeks) decreased blood glucose and serum triglycerides in alloxan induced diabetic rats.

DCI and its derivatives decreased serum insulin levels, decreased serum triglyceride levels, decreased serum cholesterol levels, decreased free and total testosterone levels, improved ovulation and increased progesterone and sex hormone binding globulin in subjects suffering from metabolic disorders such as hyperinsulinemia, hyper-triglyceridemia, dyslipidemia, hypercholesterolemia, hyperandrogenism, and anovulation. These findings were the subject matter of U.S. Pat. No. 5,906,979. Another patent (U.S. Pat. No. 6,486,127) embodied that DCI prolonged pancreatic production of insulin and, acting either alone or in combination with a lipid lowering agent, reduced serum lipids in insulin resistant human subjects. Prolonged insulin production and treatment of insulin resistance or diabetes with DCI and its derivatives either alone or in combination with the class of drugs known as sulfonylureas is also described in U.S. Pat. No. 6,492,339.

From previous studies, it has been clearly established that DCI is an effective therapeutic agent to treat insulin resistance and the concomitant metabolic disorders. In these studies it was proposed that the mechanism of action of DCI is that it is an important component of key metabolites that mediate the action of insulin. This proposal was supported by studies showing DCI deficiency in certain tissues from individuals suffering from insulin resistance or a related disease state. The recognition that a possible therapeutic mechanism for DCI resides in its ability to act as an antioxidant, in addition to its role as an insulin mediator, is novel and is, thus, an embodiment of this invention. In support of this embodiment, studies have been designed to determine the details of this antioxidant mechanism. Since it is well established that insulin resistance and CVD are often concomitant disease states and that oxidative stress is integral to endothelial dysfunction, it is a further embodiment of this invention that derivatives of DCI are useful in treating endothelial dysfunction through their ability to act as an antioxidant.

The studies to support the embodiments of this invention are directed toward determining the magnitude of the effect that DCI and a lipophilic derivative have on reversing endothelial dysfunction in diabetic animal models.

Acetylcholine induces the relaxation of aortic rings and evokes a vasorelaxant response in the endothelium of the vascular mesenteric bed. It has been shown that these endothelial-dependant effects are impaired in tissues from diabetic rats.

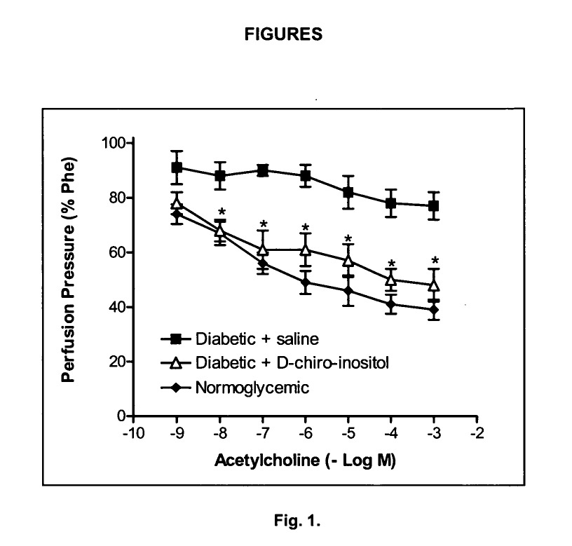

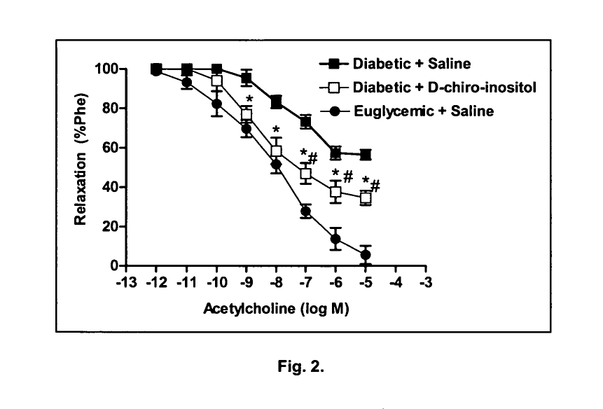

Tissues from alloxan-diabetic rats treated with DCI had a higher sensitivity and a maximal decrease in perfusion pressure of 52±6% compared to treatment with saline alone (FIG. 1). In addition, the maximal relaxant response to acetylcholine in aortic rings obtained from diabetic rats treated with saline was 57.4±4.5% compared to 100% relaxation obtained in tissues from normoglycemic rats (FIG. 2). Thus, the administration of DCI for 4-weeks was shown to be effective in improving endothelial dysfunction observed in the saline treated alloxan diabetic rats.

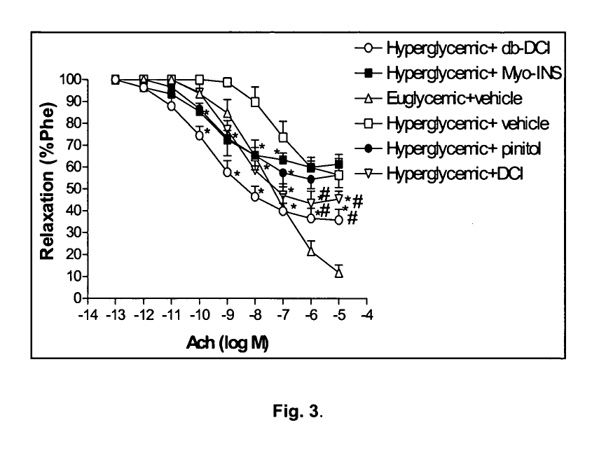

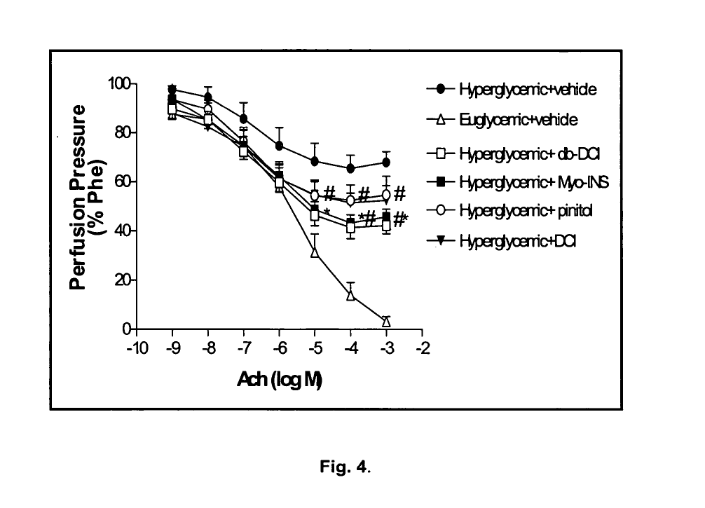

The effects of chronic (4-week) oral administration of several different inositols on macrovascular and microvascular endothelial dysfunction were compared. Alloxan-diabetic rat aortic rings were vasodilated with acetyl choline following phenylephrine vasoconstriction. Chronic oral administration of dbDCI was most effective at the lowest acetyl choline concentrations in preventing or ameliorating endothelial dysfunction in the macrovascular bed. At the intermediate concentration of acetylcholine (10-7 M) dbDCI had the greatest effect followed by DCI, pinitol and myoinositol in descending order of effect (FIG. 3). All four inositol congeners were approximately equally effective on microvascular endothelial dysfunction, with dbDCI and myoinositol perhaps slightly better than pinitol and DCI (FIG. 4).

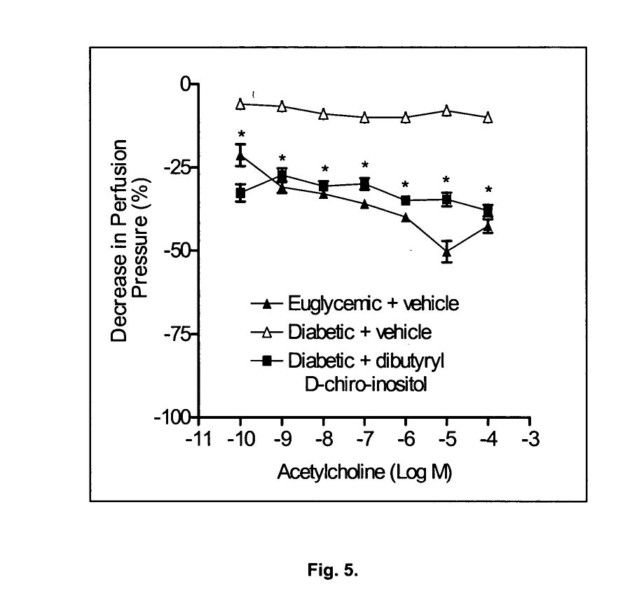

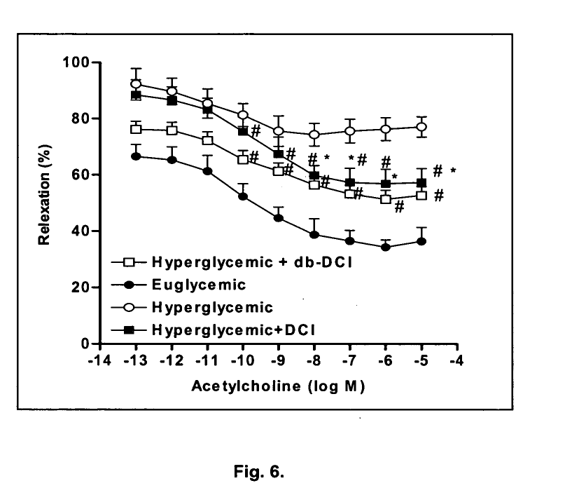

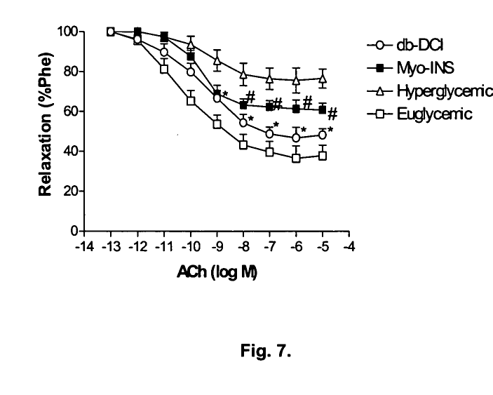

DCI also partially restored vasorelaxant effect of acetylcholine in the renal vascular bed of perfused kidneys from alloxan-diabetic rabbits. The more lipophilic derivative of DCI, 3,4-dibutyryl-D-chiroinositol (db-DCI) had a similar effect (FIG. 5). Perfusion with 1 μM db-DCI completely restored endothelial function of these diabetic rabbits as measured by the acetylcholine induced decrease in perfusion pressure (FIG. 6). In addition, acute incubation of dbDCI was more effective than myoinositol in restoring vasorelaxant effect in aortic rings of alloxan diabetic rabbits. (FIG. 7)

These data indicate that the chronic administration of DCI improves endothelial dysfunction in diabetic rats and rabbits and acute administration of DCI and dbDCI to diabetic rabbit kidneys, in vitro, reversed endothelial dysfunction suggesting a direct interaction at the endothelial cell to at least partially restore the defect. Thus it is an embodiment of this invention that the administration of therapeutic doses of dbDCI is useful in preventing or ameliorating the development of endothelial dysfunction associated with the hyperglycemia and hypertriglyceridaemia of diabetes. It is a further embodiment of this invention that administration of DCI, either as an acute treatment or as a prophylactic, will ameliorate or reverse the conditions associated with oxidative stress.

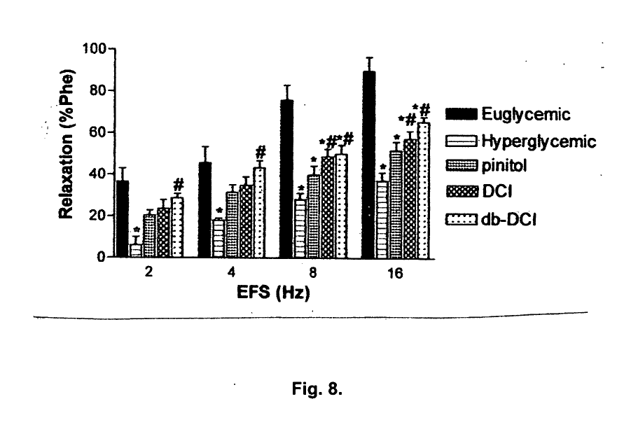

Acute incubation (60 min.) in low dose alloxan-diabetic rabbit penile corpus cavernosus with DCI, pinitol and dbDCI exhibited a restoration of vasorelaxant effect of electrical stimulation of the pudendal nerve. Almost complete restoration of the vasorelaxant effect was seen with dbDCI at EFS of 4 Hz. dbDCI had the greatest effect at all levels of electrical stimulation with pinitol and DCI have about the same effect (FIG. 8). Thus DCI, pinitol and dbDCI exhibited potential to treat erectile dysfunction. In some of the studies dbDCI showed a slight improvement over DCI. The theory behind incorporating dbDCI in the above studies was to show that by increasing the lipophilicity of the pharmacophore absorption of the therapeutic agent across the cellular membranes will be enhanced; this is consistent with the well known pharmacokinetic principles of drug absorption. With all the possible permutations of bond types, chain lengths and degree of substitution the number of different derivatives of DCI that could have been tested is extremely large. The dibutyryl derivative was selected based on a combination of the projected stability of the butyl esters in the studies and its ease of synthesis.

Currently, it is not completely understood why increasing the lipophilicity of DCI improves its performance or if other lipophilic groups would alter DCI's performance further. Although pinitol was shown to exhibit significant improvement of endothelial dysfunction in vitro, dbDCI appears to perform better. It is reasonable to expect that the nature of the bond between the lipophilic group and DCI may also have an impact on the performance of DCI. The ester linkage of dbDCI could have been replaced with an ester, ether, phosphate ester, sulfate ester, amidate, carbonate, carbamate, imidate or xanthate linkage and the resultant congeners could have performed as well, if not better, than DCI.

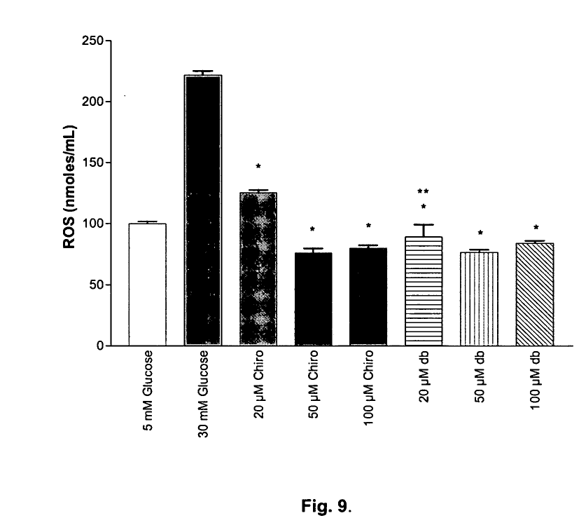

When bovine aortic endothelial cells are transferred from normal (5 mM) glucose to high glucose (30 mM), ROS in the cells increase 2 fold. When DCI or dbDCI is added to the cells ROS was reduced back to basal concentrations in a dose dependant manner. Dibutyryl-DCI had a greater effect of reducing ROS than DCI at lower concentrations (FIG. 9).

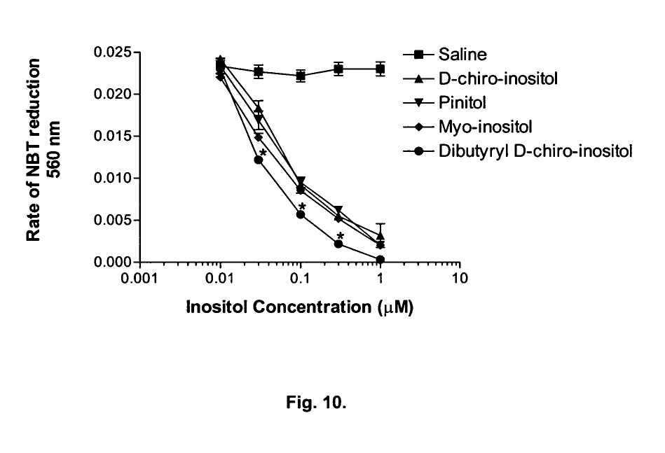

As mentioned earlier, myoinositol and its phosphate ester, phytic acid, may have multiple antioxidant mechanisms. This is supported in the experiment where DCI, dbDCI, myoinositol or pinitol added to ROS generated by xanthine/xanthine oxidase, reduced ROS in a low micromolar concentration dependant manner. It should be pointed out that dbDCI was statistically more effective than the other 3 inositols, each of which were roughly equally effective (FIG. 10).

From these data it has been shown that DCI has antioxidant properties in addition to the role it plays in mediating the action of insulin. The ability to scavenge oxyradicals, block glucose from participating in the ferrous/ferric redox reaction or to chelate iron and other metals have been proposed as the antioxidant mechanism of inositols and inositol phosphates. The ability for phytic acid to chelate metals, particularly iron, has been attributed to its ability to serve as an antioxidant. DCI only differs from myoinositol by a simple inversion at one of the chiral centers and thus it is reasonable to expect that the phosphate ester of DCI would have the same chelating properties as phytic acid and, thereby, have similar antioxidant properties as well.

It has been established that DCI and dbDCI are useful in the treatment of endothelial dysfunction and oxidative stress. Furthermore, it was discussed that the same underlying mechanism of endothelial dysfunction resulting from oxidative stress is prevalent in many other cell types and tissues. It has also been discussed that other antioxidants, such as vitamin E, are useful in the treatment of many disease states that are associated with oxidative stress. In fact, studies have shown that oxidative stress is associated with or otherwise exacerbates many diseases such as kidney disease, hepatic fibrinogenesis, Alzheimer's disease, aging, Parkinson's disease, lumbar disc degeneration, Crohn's disease, erectile dysfunction, fibriotoxicity, chronic pancreatitis, glaucoma, muscular dystrophy, fibriomyalgia, rheumatoid arthritis, amyloid lateral sclerosis, wound healing, spermatozoa damage, high blood pressure, cancer, cataracts, asthma, nonischemic cardiopathy, exercise intolerance in patients with heart failure and arterial fibrillation.

It is therefore an embodiment of this invention that DCI is useful, as a drug or a nutritional supplement, in the treatment or prevention of oxidative stress and complications resulting from oxidative stress in kidney disease, hepatic fibrinogenesis, Alzheimer's disease, aging, Parkinson's disease, lumbar disc degeneration, Crohn's disease, erectile dysfunction, fibriotoxicity, chronic pancreatitis, glaucoma, muscular dystrophy, fibriomyalgia, rheumatoid arthritis, amyloid lateral sclerosis, wound healing, spermatozoa damage, high blood pressure, cancer, cataracts, asthma, nonischemic cardiopathy, exercise intolerance in patients with heart failure and arterial fibrillation.

The synthesis of dbDCI is shown in scheme 1. The synthesis follows a standard protocol for manipulating protecting groups on the various hydroxyl groups of inositol to direct substitution of the appropriate electrophile (butyric acid for dbDCI). In the case of DCI convenient use is made of the two sets of cis-hydroxyl groups at positions 1,2 and 5,6 to prepare the diacetonide. Positions 3 and 4 are substituted with butyrate. Subsequent deprotection with acid gives the dbDCI product in overall excellent yield.

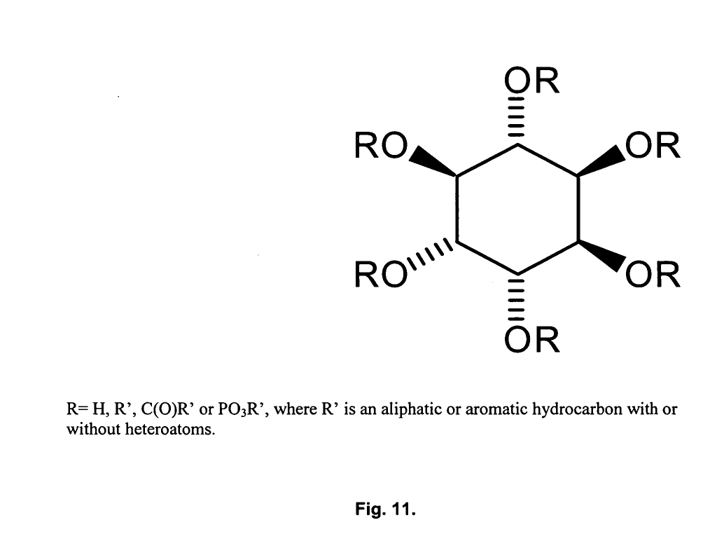

The monograph entitled Inositol Phosphates and Derivatives gives a comprehensive description of techniques used to prepare analogs of inositols. Given that dbDCI, pinitol and phytic acid are known, one skilled in the art would recognize that this and other publications support the premise that virtually any combination of ester, ether and phosphate esters of inositol is available through appropriate manipulation of protecting groups and electrophilic reagents. It is therefore an embodiment of this invention that combinations of ester, ether or phosphate esters of DCI, as represented by FIG. 11, are useful in the amelioration and treatment of endothelial dysfunction, oxidative stress and disease states related to oxidative stress including kidney disease, hepatic fibrinogenesis, Alzheimer's disease, aging, Parkinson's disease, lumbar disc degeneration, Crohn's disease, erectile dysfunction, fibriotoxicity, chronic pancreatitis, glaucoma, muscular dystrophy, fibriomyalgia, rheumatoid arthritis, amyloid lateral sclerosis, wound healing, spermatozoa damage, high blood pressure, cancer, cataracts, asthma, nonischemic cardiopathy, exercise intolerance in patients with heart failure and arterial fibrillation.

DCI or a derivative thereof, which includes but not limited to pinitol and dbDCI, used in accordance with the present invention can also comprise the active component in a pharmaceutical composition. The pharmaceutical compositions of DCI (or a derivative thereof) may be administered to any animal that would benefit therefrom, particularly humans.

These pharmaceutical compositions will be formulated and dosed in a fashion consistent with good medical practice, taking into account the clinical condition of the individual patient (especially the side effects of treatment with the active agent), the site of delivery of the composition, the method of administration, the scheduling of administration, and other factors known to practitioners. The “effective amount” of each active agent (i.e. DCI or a derivative thereof) for the purposes of the present invention is determined in view of such considerations. Those skilled in the art can readily determine empirically an appropriate “effective amount” of each active agent for a particular mammalian patient.

DCI (or a derivative thereof) according to the present invention may also be administered pharmacologically as a prodrug. The expression “prodrug” as used herein denotes a derivative of DCI which is converted to DCI in vivo by an enzymatic or chemical process but exhibits enhanced delivery characteristics and/or therapeutic value. The preparation and administration of prodrugs of saccharides, for example in the form of methylated or acetylated hydroxyl groups, is well known in the art. (Baker, D. C., et al., J. Med. Chem. 27:270-274 (1984)).

As used herein, the phrase “pharmaceutically acceptable” is intended to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “pharmaceutically acceptable carrier” is a pharmaceutically acceptable material, composition or vehicle, such as a liquid or solid filler, diluent, excipient, solvent or encapsulating material, involved in carrying or transporting the active agents of the inventive compositions from one organ, or portion of the body, to another organ, or portion of the body. Each carrier must be “acceptable” in the sense of being compatible with the other ingredients of the formulation and not injurious to the patient.

Some illustrative examples of materials which can serve as pharmaceutically acceptable carriers include, but are not limited to, the following: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the inventive pharmaceutical compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredients which can be combined with a carrier material to produce a single dosage form will generally be that amount of each active ingredient which, together, produce the desired therapeutic effect. Generally, out of one hundred percent, this amount will range from about 0.01 percent to about ninety nine percent of active ingredients, preferably from about 0.1 percent to about 90 percent, most preferably from about 1 percent to about 90 percent.

In general, the formulations are prepared by uniformly and intimately bringing into association the active ingredient with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non aqueous liquid, or as an oil in water or water in oil liquid emulsion, or as an elixir or syrup, or as pastilles (using an inert base, such as gelatin and glycerin, or sucrose and acacia) and/or as mouth washes and the like, each containing a predetermined amount of each active ingredient. The active ingredients of the inventive compositions may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents.

In the case of capsules, tablets and pills, the pharmaceutical compositions may also comprise buffering agents. Solid compositions of a similar type may also be employed as fillers in soft and hard filled gelatin capsules using such excipients as lactose or milk sugars, as well as high molecular weight polyethylene glycols and the like.

These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredients can also be in micro encapsulated form, if appropriate, with one or more of the above described excipients. Liquid dosage forms for oral administration of the inventive compositions include pharmaceutically acceptable emulsions, microemulsions, solutions, suspensions, syrups and elixirs. In addition to the active ingredients, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3 butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredients, may contain suspending agents as, for example, ethoxylated isostearyl alcohols, polyoxyethylene sorbitol and sorbitan esters, microcrystalline cellulose, aluminum metahydroxide, bentonite, agar agar and tragacanth, and mixtures thereof.

Formulations of the pharmaceutical compositions of the invention for rectal or vaginal administration may be presented as a suppository, which may be prepared by mixing the active ingredients of the present invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active ingredients. Formulations of the present invention which are suitable for vaginal administration also include pessaries, tampons, creams, gels, pastes, foams or spray formulations containing such carriers as are known in the art to be appropriate.

Dosage forms for the topical or transdermal administration of the inventive compositions include powders, sprays, ointments, pastes, creams, lotions, gels, solutions, patches and inhalants. The active ingredient may be mixed under sterile conditions with a pharmaceutically acceptable carrier, and with any preservatives, buffers, or propellants which may be required.

Ophthalmic formulations, eye ointments, powders, solutions, drops, sprays and the like, are also contemplated as being within the scope of this invention.

Pharmaceutical compositions of this invention suitable for parenteral administration comprise DCI (or a derivative thereof) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Illustrative examples of suitable aqueous and nonaqueous carriers which may be employed in the pharmaceutical compositions of the invention include, but are not limited to, the following: water; ethanol; polyols, such as glycerol, propylene glycol, polyethylene glycol, and the like, and suitable mixtures thereof; vegetable oils, such as olive oil; and injectable organic esters, such as ethyl oleate. Proper fluidity can be maintained, for example, by the use of coating materials, such as lecithin, by the maintenance of the required particle size in the case of dispersions, and by the use of surfactant.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

When the compounds of the present invention are administered as pharmaceuticals, to humans and animals, they can be given alone or as a pharmaceutical composition containing, for example, 0.01 to 99.5% (more preferably, 0.1 to 90%) of each active ingredient together in combination with at least one pharmaceutically acceptable carrier.

The preparations of the present invention may be given orally, parenterally, topically, or rectally. They are of course given by forms suitable for each administration route. Oral administration is particularly preferred.

The phrases “parenteral administration” and “administered parenterally” as used herein are intended to mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein are intended to mean the administration of a compound, drug or other material other than directly into the central nervous system, such that it enters the patient's system and, thus, is subject to metabolism and other like processes, for example, subcutaneous administration.

The inventive compositions may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the active ingredients of the present invention, which may be used in a suitable hydrated form, and/or the pharmaceutical compositions of the present invention, are formulated into pharmaceutically acceptable dosage forms by conventional methods known to those of skill in the art.

As noted, actual dosage levels of the active ingredient in the inventive pharmaceutical compositions may be varied so as to obtain an amount which is effective to achieve the desired therapeutic response for a particular patient, composition, and mode of administration, without being toxic to the patient.

The selected dosage level will depend upon a variety of factors, including, but not limited to, the following: the activity of DCI (or the derivative thereof); the route of administration; the time of administration; the rates of absorption, distribution, metabolism and/or excretion of the particular active ingredient being employed; the duration of the treatment; other drugs, compounds and/or materials used in combination with the particular active ingredients employed; the age, sex, weight, condition, general health and prior medical history of the patient being treated; and like factors well known in the medical arts.

A physician or veterinarian having ordinary skill in the art can readily determine the effective amount of the active ingredient required in the inventive pharmaceutical compositions. For example, the physician or veterinarian could start doses of the pharmaceutical composition at levels lower than that required in order to achieve the desired therapeutic effect and gradually increase the dosage until the desired effect is achieved.

If desired, the effective daily dose of the active ingredients may be administered as two, three, four, five, six or more sub doses administered separately at appropriate intervals throughout the day, optionally, in unit dosage forms.

Therapeutic compositions can be administered with medical devices known in the art. For example, a therapeutic composition of the present invention can be administered with a needleless hypodermic injection device, such as the devices disclosed in U.S. Pat. No. 5,399,163; 5,383,851; 5,312,335; 5,064,413; 4,941,880; 4,790,824, or 4,596,556. Examples of well known implants and modules useful in the present invention include: U.S. Pat. No. 4,487,603, which discloses an implantable micro infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi chamber compartments; and U.S. Pat. No. 4,475,196, which discloses an osmotic drug delivery system. Many other such implants, delivery systems, and modules are well known to those skilled in the art. Having now fully described this invention, it will be understood to those of ordinary skill in the art that the methods of the present invention can be carried out with a wide and equivalent range of conditions, formulations, and other parameters without departing from the scope of the invention or any embodiments thereof. All patents and publications cited herein are hereby fully incorporated by reference in their entirety.

The following examples illustrate the present invention without, however, limiting the same thereto.

### EXAMPLES

dbDCI Synthesis

3,4-di-butyryl-D-chiro-inositol was prepared from D-chiro-inositol, obtained from Insmed, Inc. Glen Allen, Va., in four steps. Formation of the tri-acetonide was followed by selective deprotection of the trans-acetonide and afforded D-chiro-inositol-1,2;5,6-di-acetonide in 73% overall yield. Acylation was accomplished with butyric acid, 1-ethyl-3-(3′-dimethylaminopropyl)carbodiimide (EDCI), and catalytic dimethylaminopyridine (DMAP) in 48% yield. The acetonides were then removed in AcOH/H2O (12/5, reflux, 30 min) to afford pure 3,4-di-butyryl-D-chiro-inositol in 95% yield after recrystallization from hot EtOAC. Myo-inositol and pinitol are obtained from commercial sources.

Animals.

Male Wistar rats, weighing between 230-250 g, fed regular chow, with free access to water were separated in four groups (n=10/each). The first two groups, termed normoglycemic, were treated with saline (0.1 mL/100 g/12 h) by the oral route (intragastric gavage) or untreated for four weeks. The second two groups, hyperglycemic, were treated with saline (0.1 mL/100 g/12 h) or treated with D-chiro-inositol (20 mg/kg/12 h) under the same protocol. The dose was determined from a previous dose response of acute administration of D-chiro-inositol to STZ-diabetic rats which demonstrated that 20 mg/kg was a maximal dose ( ). Diabetes was induced by a single intravenous injection of 45 mg/kg alloxan as described by Sannomiya et al., 1997 ( ). Diabetes was diagnosed 96 h after injection by blood glucose assay>200 mg/dL. All protocols were approved by the local committee and follow the Guide for the Care and Use of Laboratory Animals published by the US National Institutes of Health (NIH Publication No. 85-23, revised 1996).

Male California rabbits, weighing 2-2.5 kg, were provided by the vivarium of the Biomedicine Institute-UFC. Diabetes was induced by intraperitoneal injection of alloxan (150 mg/kg) and only animals with plasma glucose concentration above 200 mg/dl were included in the study. Age-matched euglycemic rabbits were used as controls.

Rat Aortic Thoracic Rings.

After completion of blood pressure measurements and vascular mesenteric bed collection, animals were sacrificed by exsanguination while under anesthesia. The thoracic aorta was excised and rapidly cleansed of connective tissue in Petri dishes containing warmed and gassed (95% O2 in 5% CO2) Krebs-Henseleit solution (KHS). The aorta segment was cut in small rings (≈5 mm) and mounted horizontally in 5 mL chambers for isometric recording of tension. The resting tension was adjusted to 1 g and the tissue allowed to equilibrate for 1 hour with a 15 min washout period. Tissues were kept in KHS at 37° C., gassed with the O2, CO2 mixture and isometric recordings made with a force displacement transducer connected to a four-channel polygraph (Narco Biosystems, Houston, Tex., USA). After the equilibration period, tissues were contracted with a submaximal concentration of phenylephrine (≈60% maximal response) until two similar responses were obtained in order to ensure reproducibility of the contraction curves. In the plateau phase of the last reproduced contraction, dose response curves to acetylcholine (10-1 to 10-5 M) were performed to determine endothelium-dependent relaxation. The acetylcholine dose response curve was repeated after a 60 min washout interval. All experiments were performed in the presence of indomethecin (10 μM) and guanethidine (10 μM) to prevent the production of vasoactive prostanoids. At least 6 separate experiments will be performed each in duplicate.

The administration of D-chiro-inositol (20 mg/kg/12 h; by gavage) for four weeks prevented the endothelial dysfunction observed in the control untreated diabetic group. The relaxant response induced by acetylcholine was reversed 100% of the pre-contractile state with a calculated PD2 value of 10.3 [14.5−9.5] established by phenylephrine, (p<0.05) at the lower acetylcholine concentrations, compared to the response obtained in vessels obtained from rats treated with saline. At the higher acetylcholine concentrations relaxation was partially restored to normal. (FIG. 2)

Alloxan-diabetic rat aortic rings were vasodilated with acetyl choline following phenylephrine vasoconstriction. The effects of chronic oral inositol of dibutyryl D-chiro-inositol was most effective at the lowest acetyl choline concentrations in preventing or ameliorating endothelial dysfunction, while myo-inositol, pinitol and D-chiro-inositol were all active at intermediate acetyl choline concentrations. At the highest acetyl choline concentrations dibutyryl D-chiro-inositol and D-chiro-inositol were the most effective. (FIG. 3)

Rat Arteriolar Mesenteric Bed.

Animals were maintained under anesthesia, abdomens opened and superior mesenteric arteries cannulated with PE40 polyethylene catheters at their bifurcations with the abdominal aortas. The mesentery was carefully removed by cutting close to the intestinal border with a small scissors. The pancreatic-duodenal, ileo-colic, and colic branches of the superior mesenteric artery were freed and the vascular bed excised. The vascular bed was rapidly perfused with warm KHS (37° C.; pH 7.4; gassed with 95% O2 in 5% CO2) in an open system with a constant flow of 4 ml/min maintained with a peristaltic pump. Alterations in perfusion pressure were continuously recorded in a four-channel polygraph (Narco Biosystems, Houston, Tex., USA) by means of a pressure transducer (P23 Gould Stratham, Oxnard, Calif., USA). After a 30 minute equilibration period, phenylephrine (1 to 5 μM) was infused by means of an infusion pump at a constant rate (0.1 ml/min) in order to achieve a stable perfusion pressure (100-120 mm Hg). Dose response curves were performed with vasodilators, i.e. acetylcholine (Ach; 10-9 to 10-3 M). Drugs were injected as 50 μL boluses, previously shown not to effect perfusion pressure in a side arm just above the cannula in the mesenteric artery. At least 6 separate experiments were performed each in duplicate.

The endothelium-dependent vasorelaxant response evoked by acetylcholine in the vascular mesenteric bed was impaired in tissues obtained from diabetic rats. The maximal decrease in perfusion pressure of the arteriolar mesenteric bed of normoglycemic rats was 61±3.7% (PD2=7.9 [10.8−4.9]) versus 23±5% (PD2=5.2 [5.8−3.5]) observed in tissues from 4-week alloxan-diabetic rats treated with saline. Tissues from alloxan-diabetic rats treated with D-chiro-inositol had a higher sensitivity and a maximal decrease in perfusion pressure of 52±6% (p<0.001) to acetylcholine with a calculated PD2 value of 7.4 [10.4−5.7] compared to the control group. (FIG. 1)

All 4 inositols administered orally were effective on microvascular endothelial dysfunction at the highest acetyl choline concentrations, with dibutyryl D-chiro-inositol and myo-inositol perhaps slightly better than pinitol and D-chiro-inositol. (FIG. 4)

Acute kidney and aortic ring experiments.

Kidneys and aortic rings are harvested from diabetic rabbits after 3 weeks of alloxan injection. They are perfused with KHS containing 1 μM D-chiro-inositol, pinitol, dibutyryl D-chiro-inositol, myo-inositol or saline for 1 hour. Dose response curves for acetylcholine are then performed following prior phenylephrine vasoconstriction as described. At least 6 separate experiments will be performed each in duplicate.

Diabetic rabbits were produced as above with alloxan injection. After 3 weeks kidneys were removed and perfused with 1 μM dibutyryl D-chiro-inositol, a more lipophilic derivative of D-chiro-inositol, and endothelial dysfunction evaluated. Function was restored to normal by perfusion with 1 μM dibutyryl D-chiro-inositol. (FIG. 5)

When the perfusion experiment was repeated in 3-week diabetic rabbit kidneys comparing both D-chiro-inositol (DCI) and dibutyryl D-chiro-inositol (db-DCI), function was partially restored by both compounds. (FIG. 6)

Acute rabbit kidney, aortic ring and penile corpus cavernosus experiments.

Kidneys and aortic rings were harvested from diabetic rabbits after 3 weeks of alloxan injection. The animals were sacrificed under sodium pentobarbital anesthesia (Hypnol®, 150-200 mg/kg). Aortic rings were prepared as described above. Rabbit kidneys were obtained from male New Zealand rabbits (1-1.5 kg) after cannulation of the renal artery in situ. [Thereafter the organ is rapidly perfused with warm KHS at 37° C., pH 7.4 and gassed with 95% O2 in 5% CO2 in an open system with a constant flow adjusted to yield 1 mL/g/min and maintained by a peristaltic pump. The alterations in perfusion pressure are continuously recorded in a four-channel polygraph (Narco Biosystems, Houston, Tex., USA) by means of a pressure transducer (P23 Gould Stratham, Oxnard, Calif., USA). After a 30 minute equilibration period, phenylephrine (1 to 5 μM) is infused by means of an infusion pump at a constant rate (0.1 ml/min) in order to achieve a stable perfusion pressure (100-120 mm Hg). Concentration-response curves are performed to the vasodilators, i.e. acetylcholine (Ach; 10-9 to 10-3 M). Drugs are injected as a 50 μL bolus, previously shown not to effect perfusion pressure in an arm side just above the cannula in the renal artery.] Following penectomy, the corpus cavernosum was dissected in Krebs solution and cleared of the tunica albuginea and surrounding tissues. Strips (2 cm) were mounted vertically in isometric baths (5 mL) in Krebs-Henseleit solution (37° C., pH 7.4 gassed with 95% O2 in 5% CO2) enriched with indomethecin (10 μM) and guanethedine (10 μM) in order to avoid prostanoid or neuronal norandrenergic interaction. After a 60 minute period for stabilization, tissues were precontracted with 10 μM phenylephrine in order to increase the basal tonus. Thereafter, frequency response curves to transmural electrical field stimulations (EFS-square wave pulses: 10 s train; 20 V, 0.5 ms, 2-16 Hz) were performed until two stable reproducible curves were attained. Then, frequency response curves were repeated after 60 min incubation with saline or 1 μM pinitol, DCI or db-DCI in the alloxan-diabetic rabbits. They are perfused with KHS containing 1 μM D-chiro-inositol, pinitol, dibutyryl D-chiro-inositol, myo-inositol or saline for 60 min. Dose response curves for acetylcholine are then performed following prior phenylephrine vasoconstriction as described. At least 6 separate experiments were performed each in duplicate.

The effects of acute incubation of inositols into low dose alloxan-diabetic rabbit aortic rings demonstrates that incubation of dibutyryl D-chiro-inositol in diabetic rabbit aortic rings is clearly more effective than myo-inositol at the medium and higher concentrations of acetyl choline. (FIG. 7)

Acute effect of 60 min incubation of inositols (1 μM) on the vasorelaxant effect of electrical stimulation of the pudendal nerve in the low dose alloxan-diabetic rabbit penile corpus cavernosus demonstrates that dbDCI has the greatest effect followed by DCI and pinitol. (FIG. 8)

Endothelial Cell Culture.

HMEC and HUVEC cells were grown in Gibco MCDB-131 media supplemented with 2 mM L-glutamine (Gibco), 10% fetal bovine serum (Gibco). The culture medium also contained 50,000 IU/L penicillin (Gibco), 50 mg/L streptomycin (Gibco), and 20 μg/L epidermal growth factor (Sigma). Cells were routinely grown in filter-capped Nunc T25 and T75 flasks and kept at 37°±0.5° C. in a humidified (90±1%) incubator under an air/CO2 (95%/5%) atmosphere.

Cells were re-fed 3 times per week and passaged once per week. Upon passaging, cells received a 30±3 second rinse with phosphate buffered saline pH 7.4, and then treated with Trypsin EDTA (0.05% Trypsin, 0.53 mM EDTA) (Gibco) for 2-7 minutes. The receiving cell surface was previously treated at room temperature with heated (37°±0.5° C.) 2% bovine gelatin serum (Sigma) for 20-30 minutes, and then allowed to stand at room temperature for ±30 minutes. Cell cultures were re-fed every 24±3 hours prior to seeding.

Superoxide Assay in Cells.

Bovine aortic endothelial cells were incubated in 5 mM (control) or 30 mM (hyperglycemic) glucose for 2 hours and reactive oxygen species (ROS) determined fluorometrically using the probe CM-H2DCFDA as described by Brownlee et al. Cells were loaded with 10 μM CM-H2DCFDDA, incubated for 45 min at 37°, and then analyzed in an HTS 7000 Bio assay Fluorescent Plate Reader using HT software. ROS production is reported as nmol/mL based on a standard curve produced with H2O2.

When bovine aortic endothelial cells were transferred from normal (5 mM) glucose to high glucose (30 mM), reactive oxygen species (ROS) in the cells increased 2 fold. With added D-chiro-inositol 20 μM, 50 μM, and 100 μM, ROS was reduced to basal concentrations in a dose dependent manner, with 50 μM being maximal. A similar effect was seen with dibutyryl D-chiro-inositol. Here 20 μM was already a maximal concentration. (FIG. 9)

Superoxide Assay In Vitro.

Superoxide radicals were generated by the reaction of xanthine (1 mM) and xanthine oxidase (10 μM/mL) in air-saturated potassium phosphate buffer (pH 7.8 at 25° C.). Superoxide anions were detected by nitroblue tetrazolium (NBT—50 μM) which undergoes reduction by O2-. to its formazan. The rate of reduction was monitored spectrophotometrically at 560 nm with an Ultrospec 1000 (Pharmacia Biotech, Cambridge, England). SOD-1, by scavanging O2-. Diminishes the rate of reduction of NBT. The volume was fixed at 2 mL. Each experiment was run in duplicate.

Inositols added to ROS generated by xanthine/xanthine oxidase, reduced ROS in a low micromolar concentration dependent manner. Again of interest, dibutyryl D-chiro-inositol was statistically more effective than the other 3 inositols myo-inositol, D-chiro-inositol and pinitol each of which were equally effective. (FIG. 10)

What is claimed is:

1. A method of treating endothelial dysfunction in an animal comprising administering an effective amount of 3,4-Di-O-butyryl-D-chiroinositol.

2. The method of claim 1, wherein one or more of the hydroxyl hydrogens of D-chiroinositol is substituted with a functional group represented by FIG. 11, where R represents the functional groups of ester, ether or phosphate ester.

3. A method of preventing diseases caused by or related to oxidative stress comprising administering an effective amount of D-chiroinositol.

4. The method of claim 3, comprising the prophylactic administration of an effective amount of D-chiroinositol wherein the amount of D-chiroinositol is about 3 mg/kg/day to 30 mg/kg/day.

5. The method of claim 3, wherein the diseases are kidney disease, hepatic fibrinogenesis, Alzheimer's disease, aging, Parkinson's disease, lumbar disc degeneration, Crohn's disease, erectile dysfunction, fibriotoxicity, chronic pancreatitis, glaucoma, muscular dystrophy, fibriomyalgia, rheumatoid arthritis, amyotrophic lateral sclerosis, wound healing, spermatozoa damage, high blood pressure, cancer, cataracts, asthma, nonischemic cardiopathy, exercise intolerance in patients with heart failure, reperfusion injury and arterial fibrillation.

6. The method of claim 3, wherein D-chiroinositol is an antioxidant.

7. The method of claim 6, wherein D-chiroinositol is a glucose scavenger.

8. The method of claim 6, wherein D-chiroinositol is a pro-oxidant scavenger.

9. The method of claim 6, wherein D-chiroinositol is a reactive oxygen species scavenger.

10. The method of claim 9, wherein D-chiroinositol is a peroxide radical scavenger.

11. The method of claim 10, wherein D-chiroinositol is a superoxide radical scavenger.

12. The method of claim 3, wherein one or more of the hydroxyl hydrogens of D-chiroinositol is substituted with a functional group represented by FIG. 11, where R represents the functional groups of ester, ether or phosphate ester.

13. A method of preventing diseases caused by or related to oxidative stress comprising administering an effective amount of D-pinitol.

14. The method of claim 13, comprising the prophylactic administration of an effective amount of D-pinitol wherein the amount of D-pinitol is about 3 mg/kg/day to 30 mg/kg/day.

15. The method of claim 13, wherein the diseases are kidney disease, hepatic fibrinogenesis, Alzheimer's disease, aging, Parkinson's disease, lumbar disc degeneration, Crohn's disease, erectile dysfunction, fibriotoxicity, chronic pancreatitis, glaucoma, muscular dystrophy, fibriomyalgia, rheumatoid arthritis, amyotrophic lateral sclerosis, wound healing, spermatozoa damage, high blood pressure, cancer, cataracts, asthma, nonischemic cardiopathy, exercise intolerance in patients with heart failure, reperfusion injury and arterial fibrillation.

16. The method of claim 13, wherein D-pinitol is an antioxidant.

17. The method of claim 16, wherein D-pinitol is a glucose scavenger.

18. The method of claim 16, wherein D-pinitol is a pro-oxidant scavenger.

19. The method of claim 16, wherein D-pinitol is a reactive oxygen species scavenger.

20. The method of claim 19, wherein D-pinitol is a peroxide radical scavenger.

21. The method of claim 20, wherein D-pinitol is a superoxide radical scavenger.

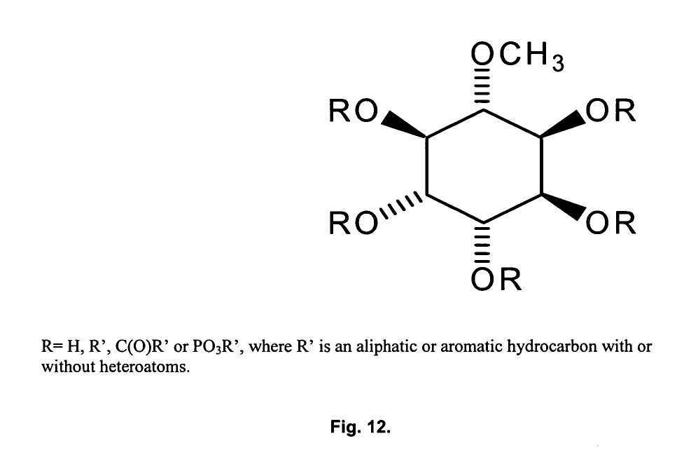

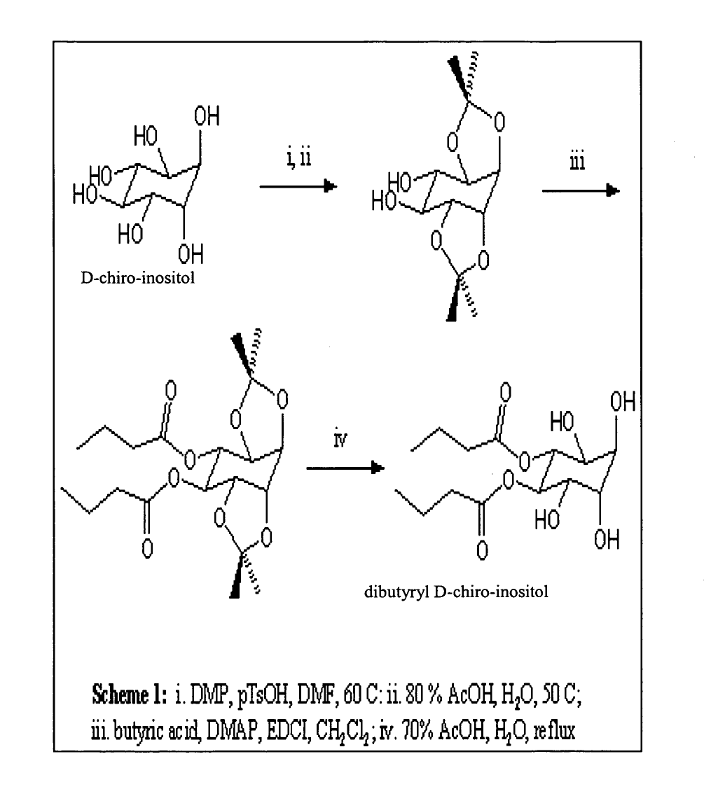

22. The method of claim 13, wherein one or more of the hydroxyl hydrogens of D-pinitol is substituted with a functional group represented by FIG. 12, where R represents the functional groups of ester, ether or phosphate ester.
